(19) United States
(12) Plant Patent Application Publication
(10) Pub. No.: US 2002/0174468 P1
(43) Pub. Date: Nov. 21, 2002

(54) Verbena plant named, 'SUNMARIRIHO'

(76) Inventor: Yasunori Yomo, Yasu-gun (JP)

Correspondence Address: BURNS, DOANE, SWECKER & MATHIS, L.L.P., P.O. Box 1404, Alexandria, VA, 22313-1404, US

(21) Appl. No.: 09/859,545

(22) Filed: May 18, 2001

(51) Int. Cl. A01H 5/00

(52) U.S. Cl. PLT/308

(57) ABSTRACT

Disclosed herein is a verbena plant which has spreading growth habit has long stems and a broad spreading area. The plant has many flowers in a spike and a great profusion of blooms. The blooming period is late April to November and the flowering duration is long. The whole bush remains in bloom for a considerable period of time. The flower size is large and the petal color of flower is yellowish white color. The plant is highly tolerant to heat, highly resistant to pests and diseases, particularly powdery mildew, and is highly resistant to rain.

### BACKGROUND OF THE VARIETY

The present invention relates to a new and distinct variety of verbena plant obtained from a crossing of Verbena hybrida ‘SUNMARISA’ (♀) and the same verbena plant variety Verbena hybrida ‘SUNMARISA’ (♂).

The verbena is a very popular plant and is used for flower bedding and potting in the summer season. There are only a few varieties of the verbena plant which have a spreading growth habit, much branching, many flowers in a spike and which have a high resistance to rain, heat, cold, and diseases. Accordingly, this invention was aimed at obtaining a new variety having a spreading growth habit, strongly branching, many flowers in a spike, large diameter flower, high tolerance to heat and cold, and resistances to diseases and pests, and also having a snow white color petal.

The new variety of verbena plant according to this invention originated from crossing of a Verbena hybrida ‘SUNMARISA’ (♀) and the same verbena plant variety Verbena hybrida ‘SUNMARISA’ (♂).

First of all, 3000 seedling were obtained in autumn of 1995, from crossing ‘SUNMARISA’ as female parent and the same verbena plant variety Verbena hybrida ‘SUNMARISA’ (♂) as a pollen parent in the May of 1995. From this crossing, 14 seedlings were selected in view of spread growth habit and petal color, and propagated by cutting, and then grown as a trial by flower bedding and planter from in the spring of 1996. Finally only one plant was selected from these 14 seedlings which were tested by flower potting and bedding in the spring of 1997 and the botanic characteristics of 14 seedlings were examined, using similar varieties ‘SUNMARIHO’ (application No. 7429 under the Seeds and Seedlings Law of Japan) and ‘CRISTAL’ for comparison. As a result, it was concluded that this verbena was distinguishable from any other variety whose existence is known to us and was uniform and stable in its characteristics. This new variety of verbena plant was named ‘SUNMARIRIHO’.

In the following description, the color-coding is in accordance with the Horticultural Color Chart of The Royal Horticultural Society, London, England (R.H.S. Color Chart), and the Inter-Society color Council-Nation Bureau of Standard Color Name (I.S.C.C.-N.B.S. Color Name). A color chart based on The Japan Color Standard for Horticultural Plant (J.H.S. Color Chart) is also added for reference.

The ‘SUNMARISA’ used as a female parent and a pollen parent in obtaining this new variety ‘SUNMARIRIHO’ are our variety which was filed in Japan in 1996 (application No. 9057 under the Seeds and Seedlings Law of Japan) and in USA in 1997 (application No. 923819 as plant patent application) and so on. ‘SUNMAIRSA’ was obtained from a crossing a Verbena hybrida ‘Amour White’ (♀) and a wild type of verbena plant Verbena peruviana (♂) native to Brazil. The main botanical characteristics of ‘SUNMARISA’ are as follows.

Plant:

Growth habit.—Spreading.

Plant extension.—Broad; 65-71 cm.

Plant height.—Very low to low; 8-9 cm.

Stem:

Diameter.—Medium; 1.7-2.2 mm.

Anthocyanin pigmentation.—Absent.

Pubescence.—Medium.

Branching.—Medium.

Subterranean stem.—Absent. When the stems contact the surface of soil, the nodes take root in the ground and the plant grows spreadingly.

Length of internode.—Medium; 1.7-2.7 cm.

Leaf:

Phyllotaxis.—Opposite.

Shape of blade.—Ovate.

Depth of blade incision.—Shallow.

Blade crenation of margin.—Serrate.

Length.—Medium; 2.9-3.6 cm.

Width.—Medium; 1.6-2.4 cm.

Color.—Dark olive green. (R.H.S. color chart No. 146A, JHS color chart No. 3707)

Pubescence.—Sparse.

Petiole.—Present.

Diameter of petiole.—Medium; 1.0-1.2 mm.

Length of petiole.—Short; 1.2-2.5 mm.

Flower:

Shape of cluster.—Funnel-shaped. (Type III)

Length of spike.—Medium; 23-44 mm.

Diameter of spike.—Medium; 40-58 mm.

Facing direction.—Upward.

Outward curvature of petal.—Slightly curved.

Diameter.—Large; 1.8-1.9 cm.

Height.—High; 2.1-2.2 cm.

Color.—Light purplish pink. (R.H.S. color chart No. 62C, JHS color chart No. 9503).

Eye color.—Present; small.

Variegation on petal.—Absent.

Color intensity.—Present.

Overlapping of petals.—Separate.

Incision of petal.—Present.

Number of petals.—Medium; 5.

Incision of calyx.—Present.

Length of calyx.—Long; 1.2-1.4 cm.

Anthocyanin pigmentation of calyx limb.—Absent.

Shape of pistil.—Two robes.

Number of stamens.—Medium.

Color of anther.—Yellow green.

Diameter of peduncle.—Thin; 0.9-1.4 mm.

Length of peduncle.—Medium; 19-45 mm.

Number of flowers.—Many; 13±2.

Reproductive organs.—1 pistil and 4 stamens.

Flower fragrance.—Absent.

Flowering time.—Early.

Flowering duration.—Long.

Physiological and ecological characteristics: High resistance to diseases and pests, particularly powdery mildew. High tolerances to heat and drought. High tolerances to rain. Medium tolerance to cold.

The ‘SUNMARIHO’ used as a comparison for this new variety ‘SUNMARIRIHO’ is our variety which was filed in Japan in 1994 (application No. 7429 under the Seeds and Seedlings Law of Japan) and in USA in 1996 (application No. 756029 as plant patent application) and so on. The main botanical characteristics of ‘SUNMARIHO’ are as follows.

Plant:

Growth habit.—Spreading.

Plant extension.—Broad. (50-80 cm).

Plant height.—Medium. (10-20 cm).

Stem:

Diameter.—Medium. (2-3 mm).

Anthocyanin pigmentation.—Present.

Pubescence.—Medium.

Prickle.—Absent.

Branching.—Abundant.

Subterranean stem.—Absent.

Length of internode.—Medium. (2.5-4.0 cm).

Leaf:

Phyllotaxis.—Opposite.

Shape of blade.—Hastate. (Type II).

Blade of incision.—Present.

Depth of brade incision.—Shallow.

Crenation of blade margin.—Crenate.

Length.—Medium. (4-6 cm).

Width.—Medium. (2-3 cm).

Color.—Dark green. (R.H.S. color chart No. 137B, JHS color chart No. 3508)

Pubescence.—Medium.

Petiole.—Present.

Petiole diameter.—Medium.

Petiole length.—Short.

Flower:

Shape of cluster.—Obconical. (Type II).

Spike length.—Medium.

Spike diameter.—Medium.

Facing direction.—Upward.

Outward curvature of petal.—Slightly curved.

Diameter.—Large.

Height.—Medium.

Color of petal.—Yellowish white (R.H.S. color chart No. 159D, JHS color chart No. 1901).

Eye color.—Absent.

Varigation.—Absent.

Color intensity.—Absent.

Overlapping of petals.—Separate.

Number of petal.—Medium.

Calyx incision.—Present.

Calyx length.—Long.

Anthocyanin pigmentation of calyx limb.—Absent.

Peduncle diameter.—Thin.

Peduncle length.—Medium.

Number of flowers.—Medium.

Flower fragrance.—Present.

Flowering time.—Late.

Flowering duration.—Long.

Physiological and ecological characteristics:

Tolerance to cold.—High.

Tolerance to heat.—High.

Resistance to diseases.—High.

Resistance to pests.—High.

‘CRISTAL’ used as a comparison for this new variety ‘SUNMARIRIHO’ is on the market. The main botanical characteristics of ‘CRISTAL’ are as follows.

Plant:

Growth habit.—Erect.

Plant extension.—Narrow. (15-20 cm).

Plant height.—High.

Stem:

Diameter.—Medium. (2.0-3.0 mm).

Anthocyanin pigmentation.—Present.

Pubescence.—Medium.

Prickle.—Absent.

Branching.—Few.

Subterranean stem.—Absent.

Length of internode.—Medium.

Leaf:

Phyllotaxis.—Opposite.

Shape of blade.—Hastate. (Type II).

Blade of incision.—Present.

Depth of brade incision.—Shallow.

Crenation of blade margin.—Serrate.

Length.—Long.

Width.—Broad.

Color.—Dark yellow green. (R.H.S. color chart No. 146A, JHS color chart No. 3508).

Pubescence.—Few.

Petiole.—Present.

Petiole diameter.—Medium.

Petiole length.—Long.

Flower:

Shape of cluster.—Obconinal. (Type II).

Spike length.—Medium.

Spike diameter.—Medium.

Facing direction.—Upward.

Outward curvature of petal.—Slightly curved.

Diameter.—Medium.

Height.—Medium.

Color of petal.—Yellowish white. (R.H.S. color chart No. 158D, JHS color chart No. 2901).

Eye color.—Absent.

Varigation.—Absent.

Color intensity.—Absent.

Overlapping of petals.—Separate.

Incision of petal.—Present.

Number of petal.—Medium.

Calyx incision.—Present.

Calyx length.—Long.

Anthocyanin pigmentation of calyx limb.—Absent.

Pistil shape.—Two robes.

Stamen number.—Medium.

Anther color.—Yellowish green.

Peduncle diameter.—Thin.

Peduncle length.—Medium.

Spike.—30-40 mm in length; and 40-55 mm in diameter.

Calyx.—1.5-2.0 cm in length.

Number of flowers.—Few.

Flower fragrance.—Absent.

Flowering time.—Late.

Flowering duration.—Medium.

Physiological and ecological characteristics:

Tolerance to cold.—Low.

Tolerance to heat.—Low.

Resistance to diseases.—Low.

Resistance to pests.—Low.

This new variety of verbena plant ‘SUNMARIRIHO’ was asexually reproduced by cutting at the aforementioned Hakushu Nursery Center of SUNTORY Ltd., residing at 29131 Torihara, Hakushu-cho, Kitakoma-gun, Yamanashi-ken, Japan, and the homogeneity and stability thereof were confirmed.

### SUMMARY OF THE VARIETY

This new variety of verbena plant has a spreading growth habit having long stems and the spreading area of plant is very broad. The plant has many branches and plentiful flowers in a spike, and great profusion of blooms. The blooming period is late April to November, and flowers are open in the rainy season and the flowering duration is long. The whole bush remains in bloom for a considerable period of time. The flower size is large and the petal color of flower is yellowish white. The plant is highly tolerant to heat and highly resistant to pests and diseases, particularly powdery mildew and is highly resistant to rain. It shows medium resistance to cold.

### BRIEF DESCRIPTION OF THE DRAWINGS

FIG. 1 is photograph giving a partial view of the new variety of verbena plant planted in the ground;

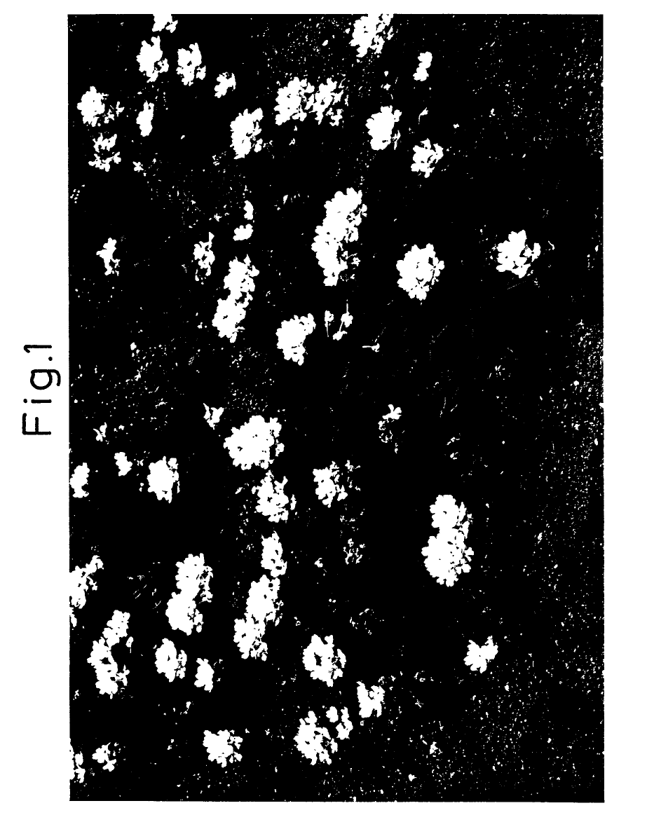

FIG. 2 is a photograph of flowers of the new variety of verbena plant.

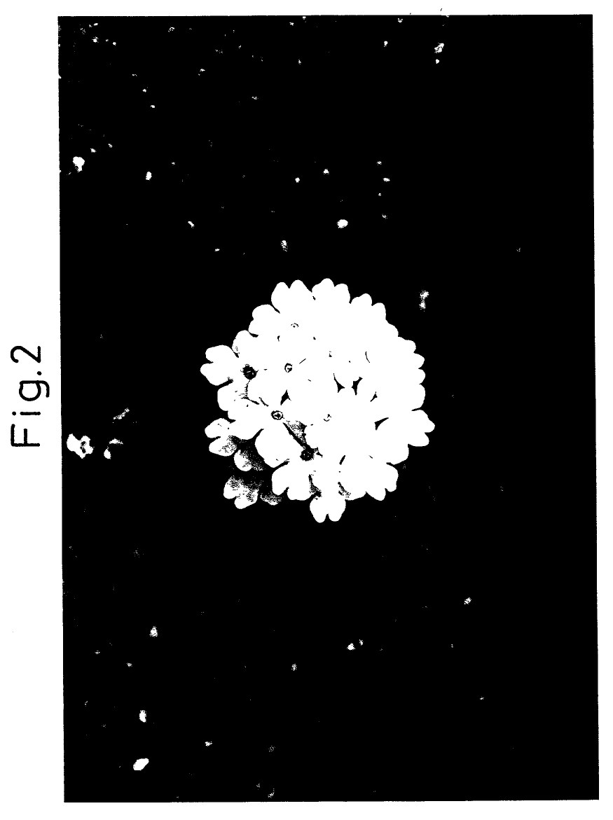

### DESCRIPTION OF THE VARIETY

The botanical characteristics of the new and distinct variety of verbena plant, ‘SUNMARIRIHO’ are as follows.

Plant:

Growth habit.—Spreading.

Plant extension.—Broad. (50-80 cm).

Plant height.—Medium. (15-20 cm).

Stem:

Diameter.—Medium. (2-3 mm).

Anthocyanin pigmentation.—Absent.

Pubescence.—Medium.

Prickle.—Absent.

Branching.—Many.

Subterranean stem.—Absent.

Length of internode.—Medium (2-4 cm).

Leaf:

Phyllotaxis.—Opposite.

Shape of blade.—Hastate. (Type II

Blade of incision.—Absent.

Crenation of blade margin.—Serrate.

Length.—Medium.

Width.—Medium.

Color.—Grayish olive green. (R.H.S. color chart No. 137A, J.H.S. color chart No. 3508).

Pubescence.—Medium.

Petiole.—Present.

Petiole diameter.—Medium.

Petiole length.—Short.

Flower:

Shape of cluster.—Funnel-shaped. (Type III).

Spike length.—Medium. (33-40 mm).

Spike diameter.—Medium to large. (50-55 mm).

Facing direction.—Upward.

Outward curvature of petal.—Slightly curved.

Diameter.—Large. (15-20 mm).

Height.—High.

Color of petal.—Yellowish white. (R.H.S. color chart No. 155D, J.H.S. color chart No. 2901).

Eye color.—Absent.

Varigation.—Absent.

Color intensity.—Absent.

Overlapping of petals.—Closed.

Incision of petal.—Present.

Number of petal.—Medium.

Calyx incision.—Present.

Calyx length.—Long.

Anthocyanin pigmentation of calyx limb.—Absent.

Pistil shape.—Two robes.

Stamen number.—Medium.

Anther color.—Yellowish green.

Peduncle diameter.—Thin.

Peduncle length.—Medium.

Number of flowers.—Many.

Flower fragrance.—Present.

Flowering time.—Medium.

Flowering duration.—Long.

Physiological and ecological characteristics:

Tolerance to cold.—Medium.

Tolerance to heat.—High.

Resistance to diseases.—High.

Resistance to pests.—High.

This new variety of verbena plant is most suitable for flower bedding and potting, particularly in planters, and is excellent for ground cover.

The plant of this new variety, ‘SUNMARIRIHO’ is presently planted and maintained at the Hakushu Nursery Center of SUNTORY Ltd., at 2913-1 Torihara, Hakushu-cho, Kitakoma-gun, Yamanashi-ken, Japan.

1. A new and distinct variety of verbena plant, substantially as herein illustrated and described, characterized particularly as to novelty by (A) having a spreading growth habit having long stems, (B) having many flowers in a spike and a great profusion blooms, the whole bush remaining in bloom for a considerable period of time, (C) having a long flowering duration,(D) the flower size is large and the petal color of flower is yellowish white color, (E) having a high resistance to rain, heat, drought, diseases and pests.
